(19) United States
(12) Plant Patent Application Publication
(10) Pub. No.: US 2010/0100992 P1
(43) Pub. Date: Apr. 22, 2010

(54) Hybrid tea rose plant named 'MEIBDEROS'

(50) ;

(75) Inventor: Alain A. Meilland, Antibes (FR)

Correspondence Address: BUCHANAN, INGERSOLL & ROONEY PC, POST OFFICE BOX 1404, ALEXANDRIA, VA, 22313-1404, US

(73) Assignee: CP DELAWARE, INC., Wilmington, DE (US)

(21) Appl. No.: 12/654,467

(22) Filed: Dec. 22, 2009

Related U.S. Application Data
(63) Continuation of application No. 12/232,455, filed on Sep. 17, 2008.
Application No. 12/232,455 is a continuation of application No. 11/520,613, filed on Sep. 14, 2006, now abandoned.
Application No. 11/520,613 is a continuation of application No. 11/066,819, filed on Feb. 28, 2005, now abandoned.
Application No. 11/066,819 is a continuation of application No. 10/114,445, filed on Apr. 3, 2002, now abandoned.

(51) Int. Cl. A01H 5/00

(52) U.S. Cl. PLT/134

(57) ABSTRACT

A new and distinct variety of Hybrid Tea rose plant is provided which abundantly forms attractive double yellow suffused with red blossoms having a very strong fragrance. The plant exhibits a semi-erect growth habit, very dense dark green foliage with a glossy aspect, and good resistance to Black Spot and Powdery Mildew. The attractive dark green foliage contrasts nicely with the blossom coloration. The new variety is particularly well suited for growing as attractive ornamentation in parks and gardens.

This is a Continuation of U.S. Plant patent application Ser. No. 12/232,455, filed Sep. 17, 2008, which is a Continuation of U.S. Plant patent application Ser. No. 11/520,613, filed Sep. 14, 2006 (now abandoned), which is a Continuation of U.S. Plant patent application Ser. No. 11/066,819, filed Feb. 28, 2005 (now abandoned), which is a Continuation of U.S. Plant patent application Ser. No. 10/114,445, filed Apr. 3, 2002 (now abandoned).

### BOTANICAL/COMMERCIAL CLASSIFICATION

Rosa hybrida/Hybrid Tea Rose

### VARIETAL DENOMINATION

cv. ‘Meibderos’

### SUMMARY OF THE INVENTION

The new variety of Rosa hybrida Hybrid Tea rose plant was created by artificial pollination wherein two parents were crossed which previously had been studied in the hope that they would contribute the desired characteristics. The female parent (i.e., the seed parent) was the ‘Purple Splendour’ variety (non-patented in the United States). The male parent (i.e., the pollen parent) was the product of the cross of the ‘Johnago’ variety (non-patented in the United States) and the ‘Meikinosi’ variety (non-patented in the United States). The ‘Meikinosi’ variety sometimes is known as the ‘Tchin-Tchin’ variety. The parentage of the new variety can be summarized as follows:

‘Purple Splendour’×(‘Johnago’×‘Meikinosi’).

The seeds resulting from the above pollination were sown and small plants were obtained which were physically and biologically different from each other. Selective study resulted in the identification of a single plant of the new variety.

It was found that the new variety of the present invention
(a) exhibits a semi-erect growth habit,
(b) abundantly forms on a continuous basis attractive double yellow suffused with red blossoms having a very strong fragrance.
(c) forms very dense dark green foliage having a glossy aspect that contrasts well with the blossom coloration, and
(d) is particularly well suited for growing as attractive ornamentation in parks and gardens.

The disease resistance is good with respect to Black Spot and Powdery Mildew, and the blossom coloration is stable.

The new variety well meets the needs of the horticultural industry and can be grown to advantage in the landscape where attractive ornamentation is desired.

The new variety can be readily distinguished from its ancestors. For instance, the ‘Purple Splendour’ variety forms blossoms of a mauve blend having approximately 26 petals per blossom on average and only a slight fragrance. The ‘Johnago’ variety forms blossoms of phlox pink with a canary yellow base of only slight fragrance. The ‘Meikinosi’ variety forms chrome yellow blossoms having approximately 30 petals on average.

The new variety has been found to undergo asexual propagation in France by a number of routes, including budding, grafting, and cuttage. Asexual propagation by the above-mentioned techniques in France has shown that the characteristics of the new variety are stable and are strictly transmissible by such asexual propagation from one generation to another.

The new variety has been named the ‘Meibderos’ variety.

### BRIEF DESCRIPTION OF THE PHOTOGRAPH

The accompanying photograph shows as nearly true as it is reasonably possible to make the same, in a color illustration of this character, typical specimens of the plant parts of the new variety. The rose plants of the new variety were approximately 18 months of age and were observed during June while budded on Rosa froebelli understock and growing outdoors at Le Cannet des Maures, Var, France. Dimensions in centimeters are indicated at the bottom of the photograph.

FIG. 1—illustrates a specimen of a young shoot;

FIG. 2—illustrates a specimen of a floral bud before the opening of the sepals;

FIG. 3—illustrates a specimen of a floral bud at the opening of the sepals;

FIG. 4—illustrates a specimen of a floral bud at the opening of the petals;

FIG. 5—illustrates a specimen of a flower in the course of opening;

FIG. 6—illustrates a specimen of an open flower—plan view—obverse;

FIG. 7—illustrates a specimen of an open flower—plan view—reverse;

FIG. 8—illustrates a specimen of a fully open flower—plan view—obverse;

FIG. 9—illustrates a specimen of a fully open flower—plan view—reverse;

FIG. 10—illustrates a specimen of a floral receptacle showing the arrangement of the stamens and pistils;

FIG. 11—illustrates a specimen of a floral receptacle showing the arrangement of the pistils (stamens removed);

FIG. 12—illustrates a specimen of a flowering stem;

FIG. 13—illustrates a specimen of a main branch;

FIG. 14—illustrates a specimen of a leaf with three leaflets—plan view—upper surface;

FIG. 15—illustrates a specimen of a leaf with five leaflets—plan view—under surface; and

FIG. 16—illustrates a specimen of a leaf with seven leaflets—plan view—upper surface.

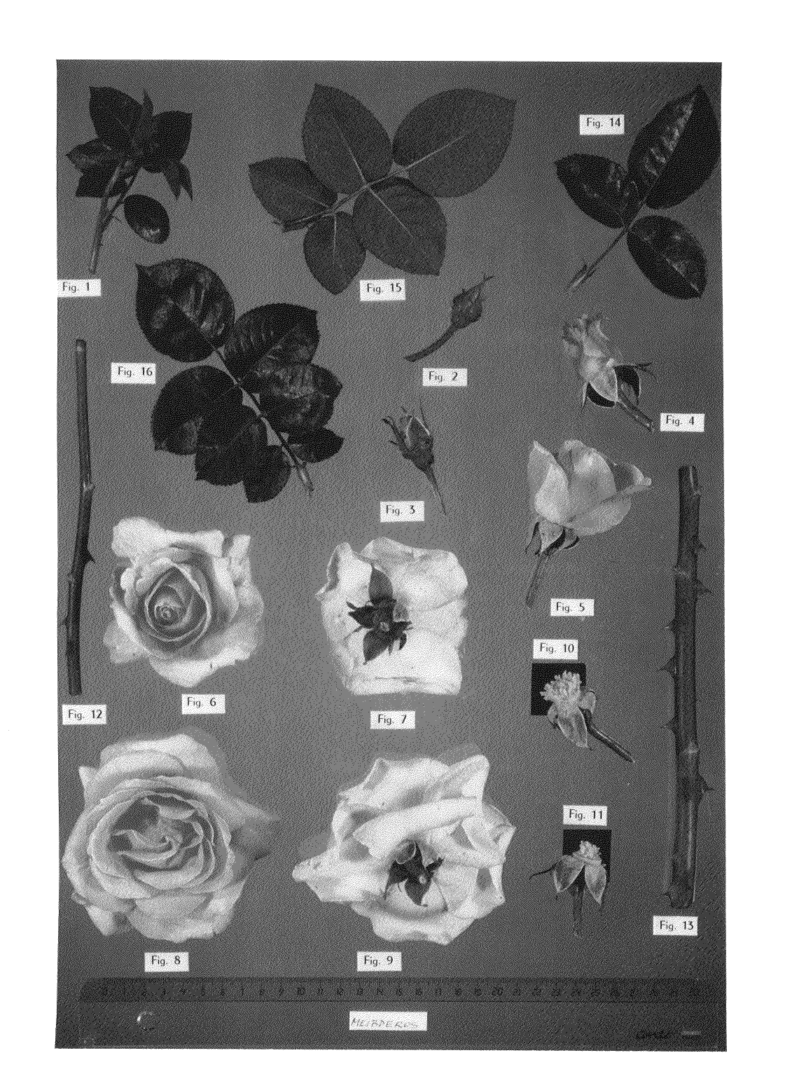

### DETAILED DESCRIPTION

The chart used in the identification of the colors is that of The Royal Horticultural Society (R.H.S. Colour Chart). The description is based on the observation of eighteen month-old plants during June while budded on Rosa froebelli understock and growing outdoors at Le Cannet des Maures, Var, France.
Class: Hybrid Tea.
Plant:
   Height.—approximately 80 to 90 cm on average at the end of the growing season.
   Width.—approximately 70 cm on average at the end of the growing season.
   Habit.—semi-erect.
Branches:
   Color.—young stems: near Yellow-Green Group 146B. adult wood: near Yellow-Green Group 146A.
   Thorns.—size: variable (as illustrated). quantity: on 15 cm of young stems approximately 6 on average more than 5 mm in length and approximately 6 on average less than 5 mm in length, and on 15 mm of adult stems approximately 9 on average more than 5 mm in length and 3 on average less than 5 mm in length. color: near Yellow-Green Group 152C slightly suffused with Greyed-Red Group 178D on young stems and on adult wood. configuration: curved downwards on the upper surface and concave under surface with a narrow and oval base.
Leaves:
   Stipules.—adnate, pectinate, rather broad.
   Petioles.—upper surface: slightly glandular with a few prickles, and near Green Group 139A in coloration. under surface: bears a few prickles, and near Yellow-Green Group 147B in coloration. length: approximately 2.2 cm for the terminal leaflet.
   Leaflets.—number: 3, 5, and 7. shape: somewhat ovate with an obtuse base and an acute tip (as illustrated). size: the terminal leaflets commonly are approximately 7 cm in length and approximately 5 cm in width on average. serration: simple, fine, and regular (as illustrated). texture: leathery. general appearance: very dense, and dark green with a glossy aspect. color (young foliage): upper surface: near Green Group 139A lightly suffused with Greyed-Red Group 178A. under surface: near Yellow-Green Group 147B suffused with Greyed-Red Group 178A. color (adult foliage): upper surface: near Green Group 139A. under surface: near Yellow-Green Group 147B.
Inflorescence:
   Number of flowers.—commonly one to three per stem.
   Peduncle.—with many tiny prickles, near Green Group 146D suffused with Greyed-Red Group 182B in coloration, and the length is approximately 3 cm on average.
   Sepals.—upper surface: tomentose and near Yellow-Green Group 147C with slight anthocyanin coloration near Greyed-Red Group 182B. under surface: slightly glandular and near Yellow-Green 147B with slight anthocyanin coloration near Greyed-Red Group 182B. configuration: elongated with a few small extensions (as illustrated).
   Buds.—shape: conical size: large (as illustrated). length: approximately 2.5 cm on average. color: upper surface: near Yellow Group 13B widely suffused with Orange Group 26A and Red Group 55A as the calyx breaks. under surface: near Yellow Group 13C widely suffused with Orange Group 26B and Red Group 55C as the calyx breaks.
   Flower.—shape: cup-shaped. diameter: approximately 10 to 11 cm on average. color (when blooming): upper surface: Yellow Group 13B towards the base and slightly suffused with Orange-Red Group 32D and Red Group 48D. The petal margin is Yellow Group 13B widely suffused with Red Group 48D. under surface: Yellow Group 13C towards the petal margin is near Red Group 49C. basal petal spot: near Green-Yellow Group 13B on the upper and under surfaces. fragrance: very strong. lasting quality: the blossoms commonly last approximately 10 to 12 days on the plant average. petal number: approximately 50 to 54 on average under normal growing conditions. petal shape: the base is obtuse and the tip is rounded and slightly denticulated and undulated. petal arrangement: imbricated; and without petaloids. petal drop: good with the petals commonly detaching cleanly. stamen number: approximately 171 on average. anthers: regularly arranged around the styles, approximately 0.5 cm in size, and near Yellow Group 13A in coloration. pollen: present and near Yellow-Orange Group 16A in coloration. filaments: near Yellow Group 13A in coloration. pistils: approximately 110 on average. stigmas: near Yellow Group 12C in coloration. styles: near Yellow Group 12D in coloration. receptacle: slightly glandular, near Yellow-Green Group 147B with slight anthocyanin coloration near Greyed-Red Group 182B, and funnel-shaped in longitudinal section. The length and width are each approximately 1 cm on average. hips: Conceal in configuration, approximately 2 to 2.5 cm in size, and the coloration is near Yellow-Green Group 146D widely suffused with Orange Group 25C.
Development:
   Vegetation.—very strong.
   Blooming.—very abundant and continuous.
   Resistance to diseases.—good with respect to Black Spot and Powdery Mildew.
   Aptitude to bear fruit.—good.

I claim:

1. A new and distinct variety of Hybrid Tea rose plant characterized by the following combination of characteristics:
(a) exhibits semi-erect growth habit,
(b) abundantly forms on a continuous basis attractive double yellow suffused with red blossoms having a very strong fragrance,
(c) forms very dense dark green foliage having a glossy aspect that contrasts well with the blossom coloration, and
(d) is particularly well suited for growing as attractive ornamentation in parks and gardens;
substantially as herein shown and described.
